(19) United States
(12) Patent Application Publication
(10) Pub. No.: US 2005/0036958 A1
(43) Pub. Date: Feb. 17, 2005

(54) Dental bleaching

(76) Inventors: Jianxun Feng, Santa Maria, CA (US); Robert L. Ibsen, Santa Maria, CA (US)

Correspondence Address: FISH & RICHARDSON P.C., 225 FRANKLIN STREET, BOSTON, MA, 02110, US

(21) Appl. No.: 10/914,835

(22) Filed: Aug. 10, 2004

Related U.S. Application Data
(60) Provisional application No. 60/494,567, filed on Aug. 13, 2003.
(60) Provisional application No. 60/559,032, filed on Apr. 5, 2004.

(51) Int. Cl. A61K 7/16; A61K 7/20; A61K 7/135

(52) U.S. Cl. 424/53; 424/62

(57) ABSTRACT

Dental bleaching compositions, and methods of using the same are disclosed.

### CROSS REFERENCE TO RELATED APPLICATIONS

This application claims the benefit of priority from U.S. Provisional Patent Application Ser. No. 60/494,567, filed Aug. 13, 2003, and also claims the benefit of priority from U.S. Provisional Patent Application Ser. No. 60/559,032, filed Apr. 5, 2004, the contents of each is hereby incorporated by reference herein in its entirety.

### TECHNICAL FIELD

This invention relates dental bleaching compositions, and to methods of using the same.

### BACKGROUND

Teeth enamel discoloration can be caused by staining, aging, or chemical damage to teeth. Some of the more common causes of teeth discoloration are medications, coffee, tea or cigarettes. People who drink significant amounts of cola soft drinks can experience similar staining. Aside from staining, there are other factors that can affect the color of an individual's teeth. Genetics can play a role. Some people have naturally brighter enamel than others. Disease can also be a factor and certain medications can cause a discoloration of the teeth.

Although white teeth have long been considered commercially desirable, the use of whitening or bleaching gels is not without problems. Tooth sensitivity can often be an unpleasant side-effect because all bleaching gels cause varying degrees of sensitivity. Prolonged exposure to teeth of bleaching gels has a number of other adverse effects such as solubilization of calcium from the enamel layer at a pH less than 5.5 with associated demoralization, penetration of intact enamel and dentin by the bleaching gel so as to risk damage to the pulpal tissue, and digestion of said bleaching/saliva solutions. But see U.S. Pat. No. 6,458,340 issued to Ibsen et al. as one notable exception to the unpleasant tooth sensitivity side effect of bleaching.

Whitening teeth through bleaching solutions is yet one means to address stains or discoloration of the teeth. These products contain peroxide(s), which actually bleach the tooth enamel. These products typically rely on carbamide peroxide as the bleaching agent and are available in several different concentrations (10%, 16%, 22%). Peroxide-containing whiteners typically come in a gel and are placed in a mouthguard. Usage regimens vary. Some products are used for about twice a day for 2 weeks, and others are intended for overnight use for 1-2 weeks.

Currently, dental whitening or bleaching, typically involves either application of a whitening gel by a dental professional in his/her office (a “chairside application”), application of a whitening gel using a tray or mouthguard that is either custom-made, preformed, or formed by the user prior to application of the gel (e.g., a “boil and bite” tray), or brushing with a dentifrice that provides a whitening agent sometimes with other hygienically-beneficial agents. These practices rely either on a costly professional application, use of a mouthguard or tray, or repeated use of a toothpaste containing whitening agents.

Previous bleaching compositions relied on water-soluble gums or polymers to provide a matrix that was used to carry and hold bleaching agents the teeth, usually in conjunction with a dental tray, mouthguard or stint. For example, bleaching agents have included peroxides, percarbonates and perborates of the alkali and alkaline earth metals or complex compounds containing hydrogen peroxide. Also, peroxide salts of the alkali or alkaline earth metals are known to be useful in whitening teeth. Thickened mixtures containing hydrogen peroxide for use as a whitening agent have been described in U.S. Pat. Nos. 5,922,307 and 6,331,292. The products described therein have a pH range from 6.0 to 10.0.

U.S. Pat. Nos. 5,922,307; 6,312,670; and 6,514,543 all entitled “Tooth bleaching compositions” are directed towards hydrogen peroxide-containing compounds that are maintained at a substantially constant pH range of 6.0-10.0 during the tooth-bleaching procedure in the presence of a calcium chelating agent.

Alternatively, thin polymeric strips that are impregnated with bleaching compounds that must be folded onto the teeth and held in place while bleaching. For example, U.S. Pat. No. 5,891,453 entitled “Delivery system for a tooth whitener using a strip of material having low flexural stiffness” is directed to a system for delivering a tooth whitening substance to a plurality of adjacent teeth, the delivery system comprising a strip of flexible material having a sufficient flexibility to form a curved shape on a plurality of adjacent teeth and a tooth whitening substance. The strip of material is readily conformable to the teeth surfaces and to interstitial tooth spaces without permanent deformation when the delivery system is placed thereagainst. The tooth whitening substance is applied to the strip of material such that when the delivery system is placed on the surface of the teeth, the substance contacts the surface providing an active onto the surface. The substance also provides adhesive attachment between the strip of material and the surface to hold the delivery system in place for a sufficient amount of time to allow the active to act upon the surface. The method of delivery includes pre-coating the strip of material or having the wearer apply substance to the strip of material and then applying the delivery system to the teeth surfaces.

U.S. Pat. No. 5,894,017 entitled “Delivery system for an oral care substance using a strip of material having low flexural stiffness” is directed to a delivery system for an oral care substance includes a strip of material having a flexural stiffness less than about 50 grams/centimeter as measured on a Handle-O-Meter per ASTM test method D2923-95. The strip of material is readily conformable without permanent deformation to a shape of a tooth and its adjoining soft tissue when the delivery system is placed thereagainst. The delivery system also includes an oral care substance applied to the strip of material such that when the delivery system is placed on a surface of the tooth and its adjoining soft tissue, the substance contacts the surface providing an active onto the surface. The substance also provides adhesive attachment between the strip of material and the surface to hold the delivery system in place for a sufficient time to allow the active to act upon the surface. A method of delivery includes pre-coating the strip of material, having the wearer apply substance to the strip of material, or having the wearer apply the substance directly to the surface before applying a strip of material.

U.S. Pat. No. 6,096,328 entitled “Delivery system for an oral care substance using a strip of material having low flexural stiffness” is directed to a system for delivering an oral care substance to the oral surface, the delivery system comprising a strip of flexible material having a sufficient flexibility to form to the contours of the oral surface. The strip of material is readily conformable to oral surfaces without permanent deformation when the delivery system is placed there against. The oral care substance is applied to the strip of material such that when the delivery system is placed on the oral surface the active contacts the surface. The oral care substance also provides adhesive attachment between the strip of material and the oral surface so as to hold the delivery system in place for a sufficient amount of time to allow the active to act upon the oral surface. Methods of delivering the oral care substance to the oral surface include pre-coating the strip of material, having the wearer apply oral care substance to the strip of material, or having the wearer apply the oral care substance directly to the oral surface and immediately applying the strip of material over the applied oral care substance.

### SUMMARY

Generally, the invention is directed toward dental bleaching compositions, and methods of using the same. Such compositions include a whitening agent, and a matrix for holding the whitening agent in such a manner that the whitening agent is released to a surface of a tooth to whiten the tooth. An example of such a matrix is a petrolatum, e.g., a solid, white petrolatum.

In one aspect, the invention features a composition for whitening one or more teeth. The composition includes a whitening agent, and a petrolatum, e.g., a white petrolatum.

In another aspect, the invention features a composition for whitening one or more teeth that includes a peroxide, a solvent, a polyoxyalkylene, a petrolatum, and a polyvinyl pyrrolidone.

In another aspect, the invention features a method for whitening one or more teeth. The method includes applying a therapeutically effective amount of a bleaching composition to one or more teeth. The bleaching composition includes a whitening agent and a petrolatum. The bleaching composition is left in contact with the one or more teeth such that the bleaching composition may whiten the one or more teeth. In a specific implementation, the applying is performed by painting the composition.

Other embodiments and/or aspects may include any one of the following or combinations of the following. The petrolatum is present in the composition from about 10 to about 90 weight percent. The petrolatum is a solid at room temperature. The petrolatum comprises white petrolatum. The whitening agent is a peroxide. The peroxide is present in the composition from about 35 to about 70 weight percent. The composition may further include a tackifying agent, for example, a polyvinyl pyrrolidone, a starch, a polyoxyalkylene, or mixtures of these materials. The tackifying agent is present in the composition from about 5 to about 30 weight percent. The composition of may further include a solvent, for example, water, glycerine, propylene glycol, a polyethylene glycol, or mixtures of these materials.

In one embodiment, the present invention provides a method for treating one or more teeth. The method includes the steps of applying a therapeutically effective amount of a bleaching composition to one or more teeth via a tray, mouthguard, stint, wand applicator or alternative means leaving the bleaching composition in contact with the one or more teeth such that the bleaching composition may whiten the tooth.

In another embodiment, the invention provides a kit for treating one or more teeth. In such an embodiment the kit may include a wand applicator having a cap portion with a threaded configuration and an applicator portion extending from the cap portion; a whitening composition container having an interior space to hold a whitening composition and having a threaded configuration complementary to the threaded configuration of the wand applicator; and a whitening composition within said whitening composition container.

Another embodiment of the invention involves a composition for whitening teeth. The composition may include one or more whitening agents present in an amount from about 3 to about 30 weight percent (3-30% hydrogen peroxide and 9-45% urea peroxide); one or more tackifying agents present in an amount from about 3 to about 50 weight percent (PVP 3-50% and petrolatum 10-90%); and one or more solubilizing agents present in an amount from about 1 to about 20 weight percent, e.g., about 1 to about 5 weight percent.

Another embodiment of the invention is directed to a composition having the following ingredients, listed by weight percent:

| INGREDIENT DESCRIPTION | % w/w |
|---|---|
| hydrogen peroxide | 5.00 |
| water | 5.00 |
| polyoxyalkylene | 5.00 |
| white petrolatum | 74.250 |
| polyvinyl pyrrolidone | 10.000 |
| flavor(s) | 0.250 |
| sodium saccharin | 0.500 |

Another embodiment of the invention is directed to a composition having the following ingredients, listed by weight percent:

| INGREDIENT DESCRIPTION | % w/w |
|---|---|
| glycerine | 30.00 |
| urea peroxide | 15.00 |
| polyoxyalkylene block Primer | 5.000 |
| white petrolatum | 44.50 |
| polyvinyl pyrrolidone | 10.00 |
| flavor(s) | 0.25 |
| sodium saccharin | 0.50 |

In another embodiment, the bleaching composition has the following formulation:

| INGREDIENT DESCRIPTION | % w/w |
|---|---|
| 50% hydrogen peroxide solution | 20.000 |
| polyoxyalkylene | 5.000 |
| white petrolatum | 74.250 |
| polyvinyl pyrrolidone | 10.000 |
| flavor(s) | 0.250 |
| sodium saccharin | 0.500 |

In another embodiment, the bleaching composition has the following formulation:

| INGREDIENT DESCRIPTION | % w/w |
|---|---|
| urea peroxide | 15.000 |
| glycerine | 10.000 |
| white petrolatum | 50.250 |
| polyvinyl pyrrolidone | 24.000 |
| flavor(s) | 0.250 |
| sodium saccharin | 0.500 |

Embodiments may have one or more of the following advantages. The bleaching compositions disclosed herein can be applied by a professional, e.g., a dentist, or by a non-dental professional, e.g., a consumer. Conveniently, the bleaching compositions can be applied by a variety of means. For example, a tray, a mouthguard or a stint may be used to apply the bleaching compositions. The bleaching compositions can also be painted directly onto the teeth, for example, by using a brush or a pen. In embodiments where the bleaching compositions are painted on directly by a user, the need for a tray, a mouthguard, or a stint is eliminated, making the bleaching process simpler and more comfortable for the user. This can improve the frequency and duration in which the bleaching compositions are used, often improving results. Moreover, the degree of sensitivity to the bleaching process itself will generally be reduced because the composition will release the bleaching agent in a controlled, timed release manner. In addition, the bleaching compositions can enable better adhesion and better resistance to dissolution, thereby enhancing the bleaching results.

The details of one or more embodiments of the invention are set forth in the accompanying drawings and the description below. Other features, and advantages of the invention will be apparent from the description and drawings, and from the claims.

### DETAILED DESCRIPTION

An embodiment uses white petrolatum as the composition's matrix that carries and holds the bleaching agent in place against the teeth. Petrolatum has been defined as a semisolid or liquid mixture of hydrocarbons derived by distillation of paraffin-base petroleum fractions. The solid form (mineral jelly) may be either water-white or pale yellow. Its chief uses are in mild ointments, cosmetics, softener in rubber mixtures and food processing (release agent in bakery products, dehydrated fruits and vegetables), protective coating (raw fruits and vegetables), and defoaming agent (beet sugar, yeast). The liquid form (white mineral oil) is used as a laxative, textile lubricant, and dispersing agent. There are three grades of both solid and liquid types with various specifications. (USP, NF, and FCC).

Polyvinyl pyrrolidone (PVP) were also optionally included for several reasons. First, PVP can enhance the adhesive force of the formulation because of its special side groups along the main chain. Second, PVP is water dissolvable and forms the second phase in the petroleum matrix, which can control the release of peroxide from the matrix. Third, PVP can be optionally added as a viscosity adjusting agent. Based on these considerations, the PVP can be around 5 to 30 percent by weight, preferably about 10 percent by weight.

The process to make the formulation as identified below is straight forward—if the solid peroxide agent is used, the peroxide can first be mixed with petrolatum, then the other ingredients defined below, such as polyvinyl pyrrolidone or glycerin can be added to the mixture. Other ingredients, such as flavors, will further be mixed. If liquid peroxide solution is used as the whitening agents, surfactant is used to make the homogenous mixture of the hydrophilic ingredients and hydrophobic ingredients.

Because of the special properties of the petrolatum based whitening gel or paste, the gel can be used directly on the teeth without the trays, mouthguards or other holders. If desired, the gel can be used with alkaline gel to further enhance the whitening effect as a two-part whitening system.

In a preferred bleaching composition, the bleaching composition has the formulation:

| INGREDIENT DESCRIPTION | % w/w |
|---|---|
| hydrogen peroxide | 5.00 |
| water | 5.00 |
| polyoxyalkylene | 5.00 |
| white petrolatum | 74.250 |
| polyvinyl pyrrolidone | 10.000 |
| flavor(s) | 0.250 |
| sodium saccharin | 0.500 |

In another implementation, the bleaching composition has the following formulation:

| INGREDIENT DESCRIPTION | % w/w |
|---|---|
| glycerine | 30.00 |
| urea peroxide | 15.00 |
| polyoxyalkylene | 5.000 |
| white petrolatum | 44.50 |
| polyvinyl pyrrolidone | 10.00 |
| flavor(s) | 0.25 |
| sodium saccharin | 0.50 |

In another embodiment, the bleaching composition has the following formulation:

| INGREDIENT DESCRIPTION | % w/w |
|---|---|
| 50% hydrogen peroxide solution | 20.000 |
| polyoxyalkylene | 5.000 |
| white petrolatum | 74.250 |
| polyvinyl pyrrolidone | 10.000 |
| flavor(s) | 0.250 |
| sodium saccharin | 0.500 |

In another implementation, the bleaching composition has the following formulation:

| INGREDIENT DESCRIPTION | % w/w |
|---|---|
| urea peroxide | 15.000 |
| glycerine | 10.000 |
| white petrolatum | 50.250 |
| polyvinyl pyrrolidone | 24.000 |
| flavor(s) | 0.250 |
| sodium saccharin | 0.500 |

The whitening composition may include a whitening agent and a tackifying agent, among other ingredients. Suitable whitening, or bleaching, agents include peroxides, such as hydrogen peroxide, carbamide peroxide or any known whitening compounds suitable for dental applications. Peroxide solutions are commercially available in varying strength, for instance H₂O₂ is available in strengths from 35 to 70%. The concentration in the composition would vary in accordance with the strength used. Therefore, the weight percent of the solution employed would be adjusted to account for the strength of solution used, as may be appreciated by one skilled in the art. In one embodiment, the whitening agent may be present in an amount from 5 to 30 weight percent.

In addition to the aforementioned whitening agents and tackifying agents, it may be desirable to include a flavor enhancing agent such as peppermint or spearmint oil. Further, a sweetener, such as saccharin, or sodium saccharin may be included in the composition. Any suitable flavor enhancing agent or a combination thereof may be included within the whitening composition. These flavoring agents may be present in amounts ranging from trace amounts 0.06 weight percent or less up to about 3 weight percent, or more as desired by the user.

Embodiments may include a variety of other ingredients. These ingredients may include a solvent, such as water, glycerin, propylene glycol, polyethylene glycol, or a variety of other compounds suitable for use as solvents for oral applications. The solvent may be present in an amount from about 10 to about 81 weight percent. The composition may also include a solubilizing agent, or emulsifying agent, such as ethoxylated castor oil. Similarly, any known solubilizing agent suitable for oral applications may be employed. Other suitable solubilizing agents include hydrogenated castor oil, or PEG 40 hydrogenated castor oil. The solubilizing agent may be present from about 1.5 to about 20 weight percent, e.g., 1.5 to about 4.25 weight percent.

A neutralizing agent, such as a hydroxide, for instance, potassium hydroxide, sodium hydroxide, or ammonium hydroxide, may also be added to the composition. Another suitable neutralizing agent is triethanolamine. The neutralizing agent is such that helps to provide a suitable pH for the overall composition. The neutralizing agent may also serve to cross-link the thickening agent or tackifying agent to form a suitable gel. In one embodiment, the neutralizing agent is present from about 0.6 to about 5.4 weight percent. The suitable pH ranges from about 5.6 to about 10, e.g., 5.6 to about 7.6.

### EXAMPLE 1

| INGREDIENT DESCRIPTION | % w/w |
|---|---|
| hydrogen peroxide | 5.00 |
| water | 5.00 |
| polyoxyalkylene | 5.00 |
| white petrolatum | 74.250 |
| polyvinyl pyrrolidone | 10.000 |
| flavor(s) | 0.250 |
| sodium saccharin | 0.500 |

### EXAMPLE 2

| INGREDIENT DESCRIPTION | % w/w |
|---|---|
| hydrogen peroxide | 5.00 |
| water | 5.00 |
| polyoxyalkylene | 5.000 |
| white petrolatum | 83.75 |
| polyvinyl pyrrolidone | 00.000 |
| flavor(s) | 0.250 |
| sodium saccharin | 1.00 |

### EXAMPLE 3

| INGREDIENT DESCRIPTION | % w/w |
|---|---|
| hydrogen peroxide | 5.00 |
| water | 5.00 |
| polyoxyalkylene | 5.00 |
| white petrolatum | 73.75 |
| polyvinyl pyrrolidone | 10.00 |
| flavor(s) | 0.25 |
| sodium saccharin | 1.00 |

### EXAMPLE 4

| INGREDIENT DESCRIPTION | % w/w |
|---|---|
| glycerine | 30.00 |
| urea peroxide | 15.00 |
| polyoxyalkylene | 5.000 |
| white petrolatum | 44.50 |
| polyvinyl pyrrolidone | 10.00 |
| flavor(s) | 0.25 |
| sodium saccharin | 0.50 |

### EXAMPLE 5

| INGREDIENT DESCRIPTION | % w/w |
|---|---|
| glycerine | 10.00 |
| urea hydrogen peroxide | 15.00 |
| ethanol | 20.00 |
| white petrolatum | 20.00 |
| polyvinyl pyrrolidone | 34.5 |
| flavor(s) | 0.50 |

### EXAMPLE 6

| INGREDIENT DESCRIPTION | % w/w |
|---|---|
| hydrogen peroxide | 8.00 |
| water | 8.00 |
| polyoxyalkylene | 10.00 |
| white petrolatum | 64.00 |
| polyvinyl pyrrolidone | 9.00 |
| flavor(s) | 0.50 |
| sodium saccharin | 0.50 |

### EXAMPLE 7

| INGREDIENT DESCRIPTION | % w/w |
|---|---|
| hydrogen peroxide | 10.00 |
| water | 10.00 |
| polyoxyalkylene | 10.00 |
| white petrolatum | 60.00 |
| polyvinyl pyrrolidone | 9.00 |
| flavor(s) | 0.50 |
| sodium saccharin | 0.50 |

### EXAMPLE 8

| INGREDIENT DESCRIPTION | % w/w |
|---|---|
| hydrogen peroxide | 15.00 |
| water | 15.00 |
| polyoxyalkylene | 15.00 |
| white petrolatum | 45.00 |
| polyvinyl pyrrolidone | 9.00 |
| flavor(s) | 0.50 |
| sodium saccharin | 0.50 |

### EXAMPLE 9

| INGREDIENT DESCRIPTION | % w/w |
|---|---|
| hydrogen peroxide | 18.00 |
| water | 18.00 |
| polyoxyalkylene | 15.00 |
| white petrolatum | 39.00 |
| polyvinyl pyrrolidone | 9.00 |
| flavor(s) | 0.50 |
| sodium saccharin | 0.50 |

### EXAMPLE 10

| INGREDIENT DESCRIPTION | % w/w |
|---|---|
| hydrogen peroxide | 10.00 |
| water | 10.00 |
| polyoxyalkylene | 10.00 |
| white petrolatum | 30.00 |
| polyvinyl pyrrolidone | 9.00 |
| flavor(s) | 0.50 |
| sodium saccharin | 0.50 |

### EXAMPLE 11

| INGREDIENT DESCRIPTION | % w/w |
|---|---|
| hydrogen peroxide | 8.00 |
| water | 8.00 |
| polyoxyalkylene | 10.00 |
| starch | 30.00 |
| white petrolatum | 34.00 |
| polyvinyl pyrrolidone | 9.00 |
| flavor(s) | 0.50 |
| sodium saccharin | 0.50 |

### EXAMPLE 12

| INGREDIENT DESCRIPTION | % w/w |
|---|---|
| hydrogen peroxide | 5.00 |
| water | 5.00 |
| polyoxyalkylene | 10.00 |
| starch | 30.00 |
| white petrolatum | 40.00 |
| polyvinyl pyrrolidone | 9.00 |
| flavor(s) | 0.50 |
| sodium saccharin | 0.50 |

A number of embodiments of the invention have been described. Nevertheless, it will be understood that various modifications may be made without departing from the spirit and scope of the invention. Accordingly, other embodiments are within the scope of the following claims.

1. A composition for whitening one or more teeth comprising:
a whitening agent; and
a petrolatum.

2. The composition of claim 1, wherein the petrolatum is present in the composition from about 10 to about 90 weight percent.

3. The composition of claim 1, wherein the petrolatum is a solid at room temperature.

4. The composition of claim 1, wherein the petrolatum comprises white petrolatum.

5. The composition of claim 1, wherein the whitening agent is a peroxide.

6. The composition of claim 5, wherein the peroxide is present in the composition from about 35 to about 70 weight percent.

7. The composition of claim 1, further comprising a tackifying agent.

8. The composition of claim 7, wherein the tackifying agent is selected from the group consisting of a polyvinyl pyrrolidone, a starch, a polyoxyalkylene, and mixtures thereof.

9. The composition of claim 7, wherein the tackifying agent is present in the composition from about 5 to about 30 weight percent.

10. The composition of claim 1, further comprising a solvent.

11. The composition of claim 10, wherein the solvent is selected from the group consisting of water, glycerine, propylene glycol, polyethylene glycol, and mixtures thereof.

12. A composition for whitening one or more teeth comprising:
a peroxide;
a solvent;
a polyoxyalkylene;
a petrolatum; and
a polyvinyl pyrrolidone.

13. A method for whitening one or more teeth, comprising:
applying a therapeutically effective amount of a bleaching composition to one or more teeth, the bleaching composition comprising a whitening agent and a petrolatum; and
leaving the bleaching composition in contact with the one or more teeth such that the bleaching composition may whiten the one or more teeth.

14. The method of claim 13, wherein the applying is performed by painting the bleaching composition the one or more teeth.
